(19) United States
(12) Patent Application Publication
(10) Pub. No.: US 2026/0070994 A1
(43) Pub. Date: Mar. 12, 2026

(54) CD25 SINGLE-DOMAIN ANTIBODY, HUMANIZED ANTIBODY AND PREPARATION METHOD THEREOF

(71) Applicant: Passion Technology (Zhuhai) Partnership (Limited Partnership), Zhuhai (CN)

(72) Inventors: Junshen ZHANG, Zhuhai (CN); Kejia LIN, Zhuhai (CN); Min HUANG, Zhuhai (CN)

(73) Assignee: Passion Technology (Zhuhai) Partnership (Limited Partnership), Zhuhai (CN)

(21) Appl. No.: 19/206,327

(22) Filed: May 13, 2025

Related U.S. Application Data
(63) Continuation of application No. PCT/CN2023/104784, filed on Jun. 30, 2023.

(30) Foreign Application Priority Data
Dec. 22, 2022 (CN) 2022116669373

(51) Int. Cl. C07K 16/28

(52) U.S. Cl. CPC C07K 16/2866; C07K 2317/24; C07K 2317/569; C07K 2317/732; C07K 2317/76; C07K 2317/92

(57) ABSTRACT

The present invention discloses a CD25 single-domain antibody, a humanized antibody and a preparation method thereof. For the CD25 single-domain antibody, a specific amino acid sequence is SEQ ID NO: 1. The non-IL-2 blocking humanized anti-CD25 single-domain antibody obtained in the present invention, in addition to having the function of specifically binding to CD25, does not interfere with IL-2 signaling, does not affect the activation function of IL-2 on T cells, reduces immunotoxic reactions, which can meet the needs of clinical applications and provides strong support for clinical applications. At the same time, antibodies and antigen-binding portions thereof including such a sequence can be used in pharmaceutical compositions and treatment methods, especially for the treatment of cancer.

### CROSS-REFERENCE TO RELATED APPLICATIONS

The application claims priority to Chinese patent application No. 2022116669373, filed on Dec. 22, 2022, the entire contents of which are incorporated herein by reference.

### SEQUENCE LISTING

The sequence listing xml file submitted herewith, named “SeqList.xml”, created on May 8, 2025, and having a file size of 23,599 bytes, is incorporated by reference herein.

### TECHNICAL FIELD

The present invention relates to the technical field of immunology, and particularly to a CD25 single-domain antibody, a humanized antibody and a preparation method thereof. The provided CD25 antibody can specifically bind to CD25, especially the human CD25 protein, without blocking the binding of interleukin-2 (IL-2) to CD25 or the IL-2 signaling pathway. Based on this, the antibody can effectively eliminate and deplete Tregs, especially in the tumor microenvironment, without affecting the proliferative activity of T cells.

### BACKGROUND

CD25 is one of the potential molecular targets for the depletion of regulatory T cells (Tregs). CD25, also known as the alpha chain of the high-affinity interleukin-2 receptor (IL-2Rα), is highly expressed on Treg cells and is either absent or expressed at low levels on T effector cells. Therefore, it is an effective target for Treg depletion. Effectively eliminating Tregs with CD25 antibodies is an effective way to relieve immune suppression and activate the immune response. Developing a CD25 antibody with excellent performance can provide more options for clinical anti-tumor treatment.

### SUMMARY

The technical problem to be solved by the present invention is to provide a CD25 single-domain antibody, a humanized antibody and a preparation method thereof, which provides strong support for clinical applications.

The technical solutions of the present invention are as follows: a CD25 single-domain antibody is provided, where a specific amino acid sequence is SEQ ID NO: 1.

The present invention further provides a plurality of CD25 single-domain antibodies, where a specific amino acid sequence is any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

The present invention further provides a humanized antibody, where after humanization modification based on a CD25 single-domain antibody with an amino acid sequence of SEQ ID NO: 1, a specific amino acid sequence is any one of SEQ ID NO: 18 and SEQ ID NO: 19.

The present invention further provides a humanized antibody, where after humanization modification based on a CD25 single-domain antibody with an amino acid sequence of SEQ ID NO: 1, a specific amino acid sequence is any one of SEQ ID NO: 20, SEQ ID NO: 21, SEQ ID NO: 22, and SEQ ID NO: 23.

The present invention further provides a method for preparing a humanized CD25 single-domain antibody, including steps of:
Step 1: preparing a CD25 antigen;
Step 2: immunizing a Lama pacos with the CD25 antigen and detecting an immune titer;
Step 3: isolating a Lama pacos PBMC and obtaining a VHH antibody fragment;
Step 4: detecting affinity of a candidate antibody;
Step 5: performing a functional experiment on the candidate antibody;
Step 6: performing humanization modification of the antibody;
Step 7: detecting ADCC of a candidate humanized antibody;
Step 8: detecting binding affinity of the candidate humanized antibody to a target protein;
Step 9: performing an FACS experiment to detect binding of the candidate humanized antibody to an antigen-bearing cell; and
Step 10: performing an experiment of blocking an IL-2 signal by the candidate humanized antibody.

The method further includes: an FACS experiment for antigen-cell binding, a functional experiment on ADCC of the candidate antibody, and an experiment of not interfering with the binding of the ligand IL-2.

By adopting the above solutions, the present invention provides a CD25 single-domain antibody, a humanized antibody and a preparation method thereof. The obtained CD25 single-domain antibody and humanized antibody have good binding performance to the target antigen, which can meet the needs of clinical applications and provides strong support for clinical applications. The provided anti-CD25 antibody can specifically bind to CD25, especially the human CD25 fragment, but does not block the binding of interleukin-2 (IL-2) to CD25 or IL-2 signaling. The antibody can effectively deplete Tregs, especially in the tumor microenvironment. The non-IL-2 blocking humanized anti-CD25 single-domain antibody obtained in the present invention, in addition to having the function of specifically binding to CD25, does not interfere with IL-2 signaling, does not affect the activation function of IL-2 on T cells, reduces immunotoxic reactions, which can meet the needs of clinical applications and provides strong support for clinical applications. At the same time, antibodies and antigen-binding portions thereof including such a sequence can be used in pharmaceutical compositions and treatment methods, especially for the treatment of cancer.

### BRIEF DESCRIPTION OF DRAWINGS

FIG. 1 is an image showing results of SDS-PAGE detection of expression of an antigen CD25-His recombinant protein;

FIG. 2 is an image showing results of two-round Nest PCR using primers for amplifying a CD25 single-domain antibody;

FIG. 3 is a diagram showing results of sequencing 20 randomly selected monoclonal clones;

FIG. 4 is an image showing results of SDS-PAGE detection of a molecular weight of an antibody;

FIG. 5 shows detection of affinity between antigens and antibodies by ELISA;

FIG. 6 shows results of FACS detection of binding of single-domain antibodies to CD25-overexpressing cell lines;

FIG. 7 shows further FACS detection of binding EC₅₀s after selecting antibody clones with good binding effects;

FIG. 8 shows conversion of fluorescence values and EC₅₀s based on FIG. 7;

FIG. 9 shows results of ELISA detection of blocking of binding between a target protein and its ligand IL-2 by candidate antibodies;

FIG. 10 shows preliminary FACS detection of binding abilities of humanized antibodies to antigen-overexpressing cells;

FIG. 11 shows detection of affinity between humanized antibodies and antigens after gradient dilution by ELISA;

FIG. 12 shows FACS detection of binding effects of two humanized antibodies with best binding effects selected according to FIG. 10 to antigen-overexpressing cells after gradient dilution;

FIG. 13 shows EC₅₀s calculated based on FIG. 12;

FIG. 14 shows results of ADCC detection of candidate humanized antibodies;

FIG. 15 shows results of BLI detection of protein-protein interaction between antigens and antibodies; and

FIG. 16 is a graph showing results of an ELISA IL-2 blocking experiment of candidate humanized antibodies.

### DETAILED DESCRIPTION OF THE EMBODIMENTS

The present invention will be described in detail below in conjunction with the accompanying drawings and specific examples.

### I. EXPERIMENTS AND RESULTS

### 1. Preparation of Recombinant CD25-his Protein and ELISA Detection

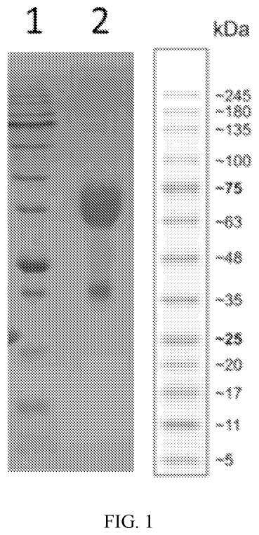

Please refer to FIG. 1. The CD25-His recombinant protein was detected by SDS-PAGE. In this figure, 1 represents a Protein MW marker, and 2 represents a CD25-His recombinant protein. The detection was performed according to the following table.

| Positive | |
|---|---|
| antibody | CD25-His |

| | | |
|---|---|---|
| 10 μg/ml | 2.653 | 2.713 |
| 2 μg/ml | 2.691 | 2.619 |
| 0.4 μg/ml | 2.757 | 2.657 |
| PBS | 0.235 | 0.195 |

With the positive control antibody as the detection antibody, the CD25-His recombinant protein at a concentration of 2 μg/ml was coated on the ELISA plate. The positive control antibody diluted in a five-fold gradient was used as the primary antibody, and the secondary antibody was Protein A-HRP. According to the ELISA detection results, the CD25-His recombinant protein could bind to the positive control antibody and thus could be used for the immunization of Lama pacos and the screening of antibodies.

### 2. ELISA Detection of Immune Titer

The detection was performed according to the following table.

Serum

| dilution | | | Second | | Third | | Fourth | |
|---|---|---|---|---|---|---|---|---|
| gradient | Background serum | | immunization | | immunization | | immunization | |
| 1:1K | 0.804 | 0.815 | 0.933 | 0.87 | 2.63 | 2.852 | 2.496 | 2.595 |
| 1:2K | 0.412 | 0.407 | 0.629 | 0.587 | 2.651 | 2.626 | 2.464 | 2.575 |
| 1:4K | 0.365 | 0.378 | 0.474 | 0.471 | 2.662 | 2.65 | 2.486 | 2.524 |
| 1:8K | 0.364 | 0.335 | 0.289 | 0.398 | 2.344 | 2.259 | 2.109 | 2.156 |
| 1:16K | 0.321 | 0.326 | 0.301 | 0.387 | 1.852 | 1.857 | 1.512 | 1.714 |
| 1:32K | 0.297 | 0.253 | 0.311 | 0.354 | 1.216 | 1.176 | 1.069 | 1.082 |
| 1:64K | 0.198 | 0.221 | 0.276 | 0.243 | 0.728 | 0.725 | 0.576 | 0.634 |
| PBS | 0.198 | 0.248 | 0.268 | 0.216 | 0.082 | 0.082 | 0.066 | 0.068 |

Result analysis: the serum of Lama pacos after immunization was taken, and limited dilution was performed according to the dilution gradient shown in the figure. ELISA detection was performed using a 96-well plate pre-coated with CD25-His antigen. According to the ELISA detection results, the immune serum bound to the target protein, and the OD450 value changed in a gradient with the serum dilution gradient, which indicated that the immunization was successful. 100 mL of peripheral blood was collected, and a single-domain antibody phage display library was established.

### 3. Amplification of VHH Fragment

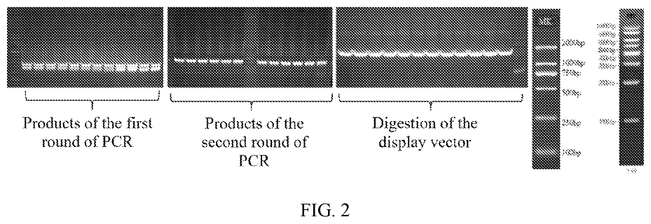

The total RNA was extracted from the peripheral blood of Lama pacos, and after reverse transcription into cDNA, two rounds of Nest PCR were performed using the amplification primers for the single-domain antibody. FIG. 2 is an image showing the results of PCR products by agarose gel electrophoresis. In the first round of PCR, two PCR bands of about 1000 bp and 750 bp were obtained. The 750-bp fragment was recovered from the gel as the template for the second round of PCR. In the second round of PCR, a band of about 450 bp was obtained, which was the VHH fragment. The VHH fragment was digested with SfiI enzyme and then subcloned into the phage display vector pComF. The ligation product was transformed into competent Escherichia coli cells by electroporation, and a single-domain antibody phage display library was constructed.

### 4. Diversity Analysis of Phage Display Library

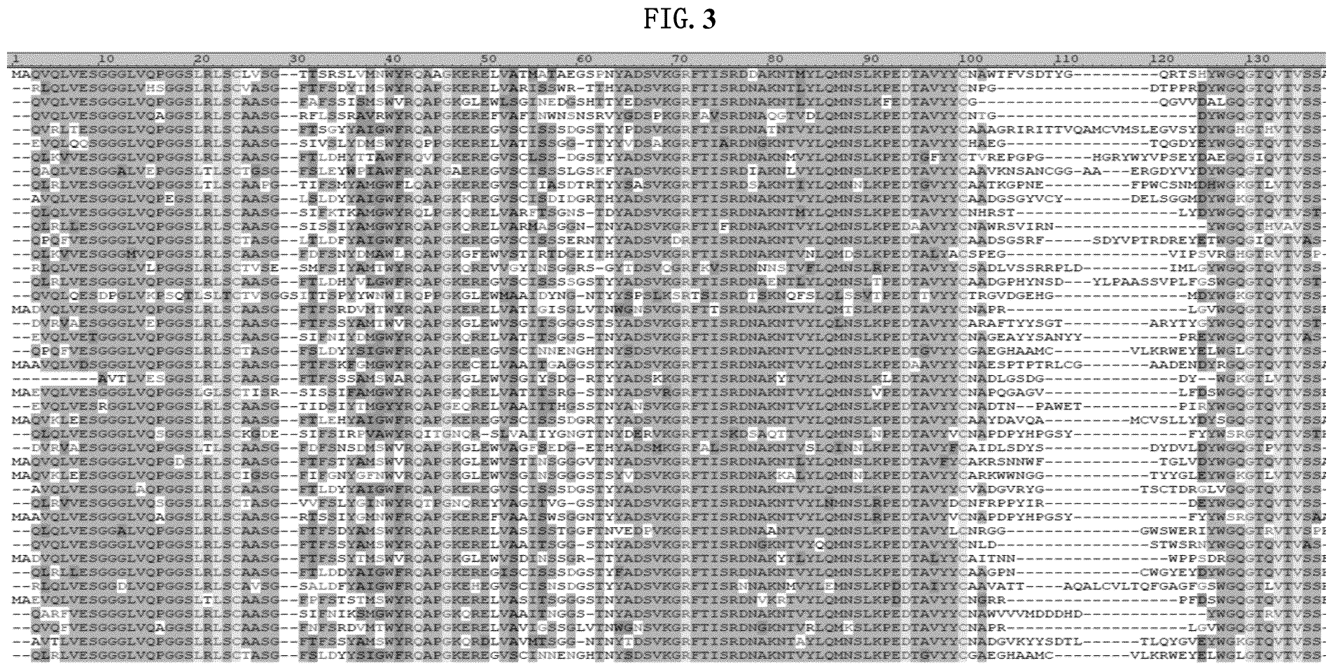

The library capacity of the phage display library was calculated to be 2.7×10⁸. 20 monoclonal clones were randomly selected from each phage display library for sequencing, and the diversity of the constructed phage display library was analyzed. As shown in FIG. 3, according to the comparison of the sequencing results, the empty vector rate and the antibody repetition rate of the phage display library were not higher than 10%.

### 5. Affinity Detection of Candidate Antibodies

5.1 The detection of the affinity between the candidate antibodies and the CD25 antigen was performed by ELISA, as shown in the following table.

| Volume of | | | | | | | | | | | | |
|---|---|---|---|---|---|---|---|---|---|---|---|---|
| transfection | | | | | | | | | | | | |
| supernatant | | | | | | | | | | | | |
| (μL) | A04 | | D07 | | D01 | | G10 | | A05 | | A08 | |
| 0 | 4.392 | 4.429 | 4.513 | 4.417 | 4.392 | 4.391 | 4.446 | 4.412 | 4.399 | 4.445 | 4.414 | 4.403 |
| 5 | 4.326 | 4.369 | 4.421 | 4.425 | 4.354 | 4.318 | 4.410 | 4.370 | 4.358 | 4.433 | 4.378 | 4.348 |
| 25 | 4.378 | 4.349 | 4.398 | 4.402 | 4.355 | 4.315 | 4.397 | 4.326 | 4.415 | 4.358 | 4.40 | 4.292 |
| 50 | 4.369 | 4.392 | 4.438 | 4.433 | 4.385 | 4.388 | 4.349 | 4.364 | 4.389 | 4.405 | 4.416 | 4.407 |
| 100 | 4.328 | 4.306 | 4.355 | 4.335 | 4.304 | 4.294 | 4.346 | 4.279 | 4.374 | 4.334 | 4.322 | 4.283 |
| PBS | 0.25 | 0.247 | 0.241 | 0.264 | 0.235 | 0.268 | 0.29 | 0.307 | 0.285 | 0.267 | 0.258 | 0.255 |

| Volume of | | | | | | | | | | | | |
|---|---|---|---|---|---|---|---|---|---|---|---|---|
| transfection | | | | | | | | | | | | |
| supernatant | | | | | | | | | | | | |
| (μL) | B04 | | E08 | | E07 | | D09 | | B10 | | C05 | |
| 0 | 4.292 | 4.418 | 4.422 | 4.377 | 4.493 | 4.48 | 4.471 | 4.466 | 4.391 | 4.358 | 4.364 | 4.37 |
| 5 | 4.365 | 4.352 | 4.404 | 4.341 | 4.538 | 4.513 | 4.489 | 4.477 | 4.383 | 4.383 | 4.329 | 4.352 |
| 25 | 4.252 | 4.368 | 4.357 | 4.345 | 4.508 | 4.448 | 4.399 | 4.394 | 4.305 | 4.342 | 4.337 | 4.31 |
| 50 | 4.342 | 4.383 | 4.392 | 4.383 | 4.607 | 4.52 | 4.525 | 4.507 | 4.346 | 4.384 | 4.375 | 4.359 |
| 100 | 4.12 | 4.262 | 4.328 | 4.272 | 4.458 | 4.441 | 4.444 | 4.443 | 4.226 | 4.266 | 4.245 | 4.193 |
| PBS | 0.2 | 0.25 | 0.228 | 0.246 | 0.245 | 0.277 | 0.288 | 0.258 | 0.252 | 0.28 | 0.302 | 0.335 |

| Volume of | | | | | | | | | | | | |
|---|---|---|---|---|---|---|---|---|---|---|---|---|
| transfection | | | | | | | | | | | | |
| supernatant | | | | | | | | | | | | |
| (μL) | C10 | | A11 | | A07 | | D08 | | D04 | | PBS | |
| 0 | 4.253 | 4.337 | 4.319 | 4.289 | 4.376 | 4.345 | 4.297 | 4.304 | 4.236 | 4.231 | 4.25 | 4.264 |
| 5 | 4.261 | 4.313 | 4.319 | 4.333 | 4.371 | 4.362 | 4.361 | 4.307 | 4.244 | 4.161 | 4.382 | 4.369 |
| 25 | 4.219 | 4.317 | 4.327 | 4.255 | 4.321 | 4.292 | 4.284 | 4.28 | 4.193 | 4.192 | 4.349 | 4.348 |
| 50 | 4.277 | 4.382 | 4.369 | 4.319 | 4.328 | 4.221 | 4.326 | 4.315 | 4.188 | 4.269 | 4.417 | 4.367 |
| 100 | 4.202 | 4.258 | 4.267 | 4.224 | 4.221 | 4.21 | 4.228 | 4.209 | 4.136 | 4.182 | 4.305 | 4.26 |
| PBS | 0.222 | 0.241 | 0.228 | 0.232 | 0.213 | 0.238 | 0.353 | 0.255 | 0.286 | 0.303 | 0.241 | 0.227 |

### ELISA Binding EC₅₀ Detection

According to the preliminary ELISA binding test results and the sequences of the candidate antibodies, several candidate antibody clones were selected to construct eukaryotic expression vectors, and recombinant antibodies were prepared for ELISA Binding EC₅₀ detection.

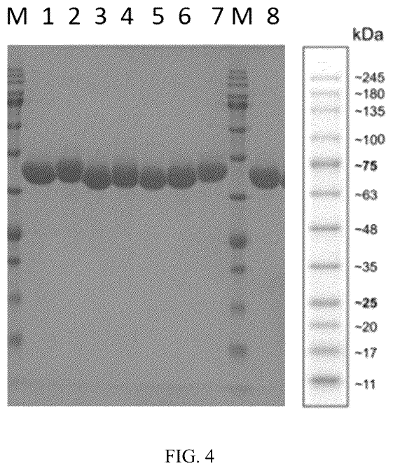

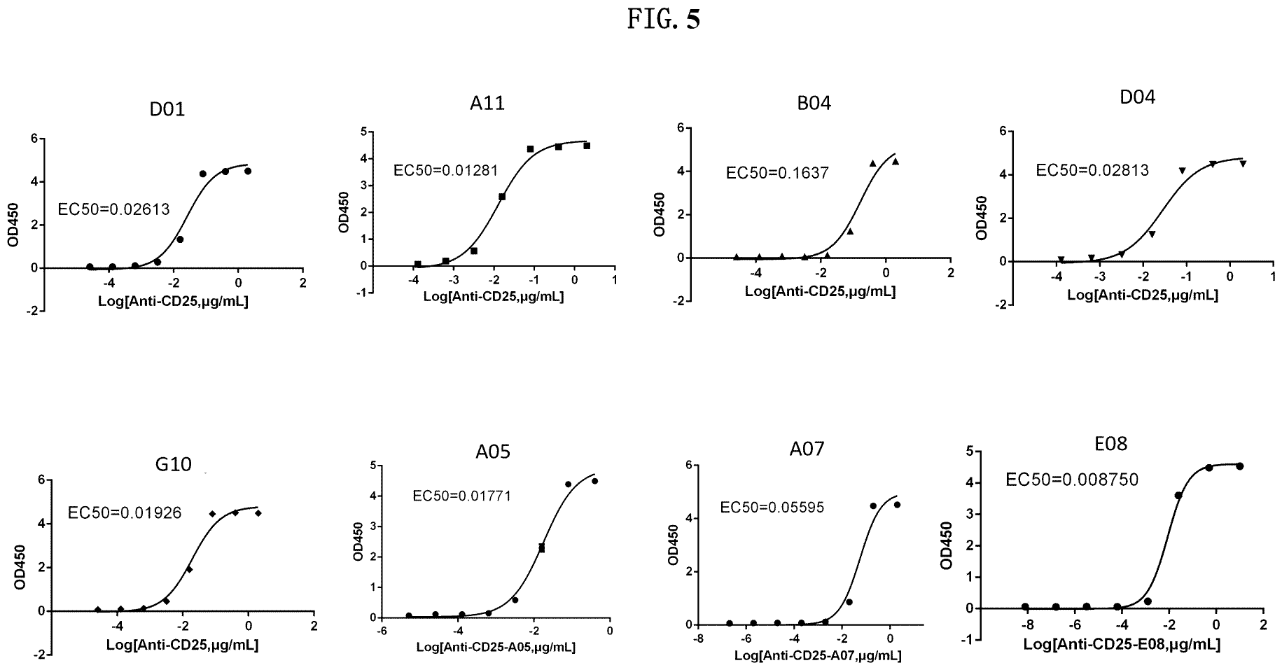

After the preparation of the recombinant antibodies, the antibody purity and the molecular weight of the antibodies were detected by adopting SDS-PAGE. The results are as shown in FIG. 4 and FIG. 5. In the figure, M: Marker, 1: clone A05, 2: clone A07, 3: clone A11, 4: clone B04, 5: clone D04, 6: clone E08, 7: clone G10, and 8: clone D01.

The CD25-His protein was coated on a 96-well microplate. After the candidate antibodies were transiently transfected into 293F cells for 48 hours, the supernatant was collected and ELISA detection was performed. The results showed that the candidate antibodies could bind to the target antigen with high affinity, and the binding EC₅₀s were equivalent to or better than that of the positive control antibody.

### 5.2 FACS Detection

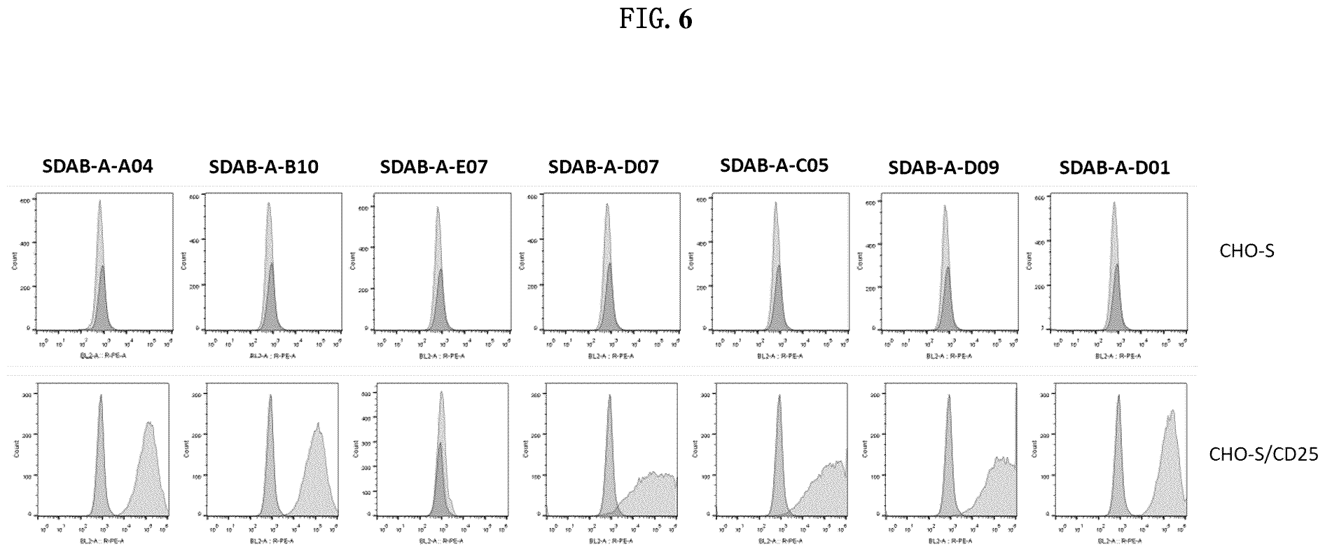

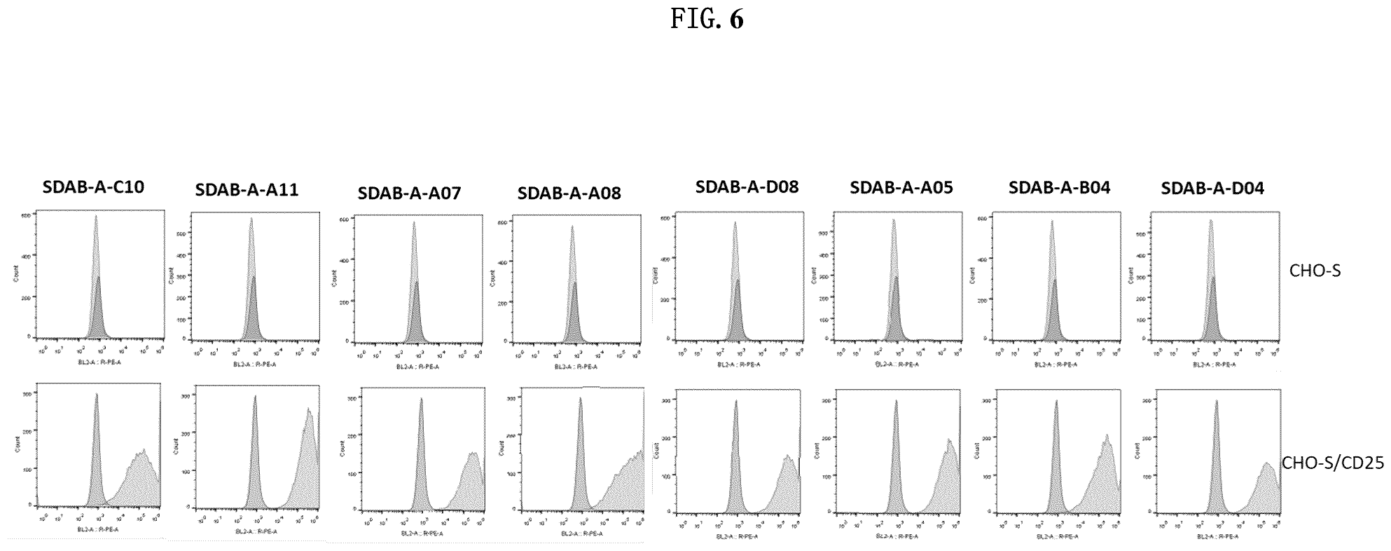

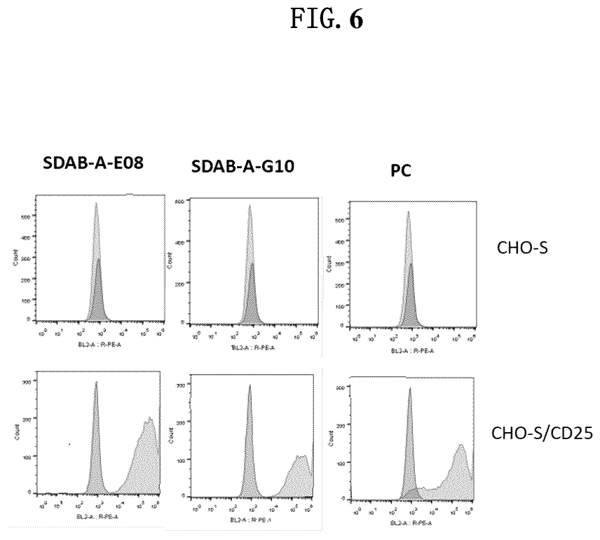

The results of the preliminary FACS detection are as shown in FIG. 6.

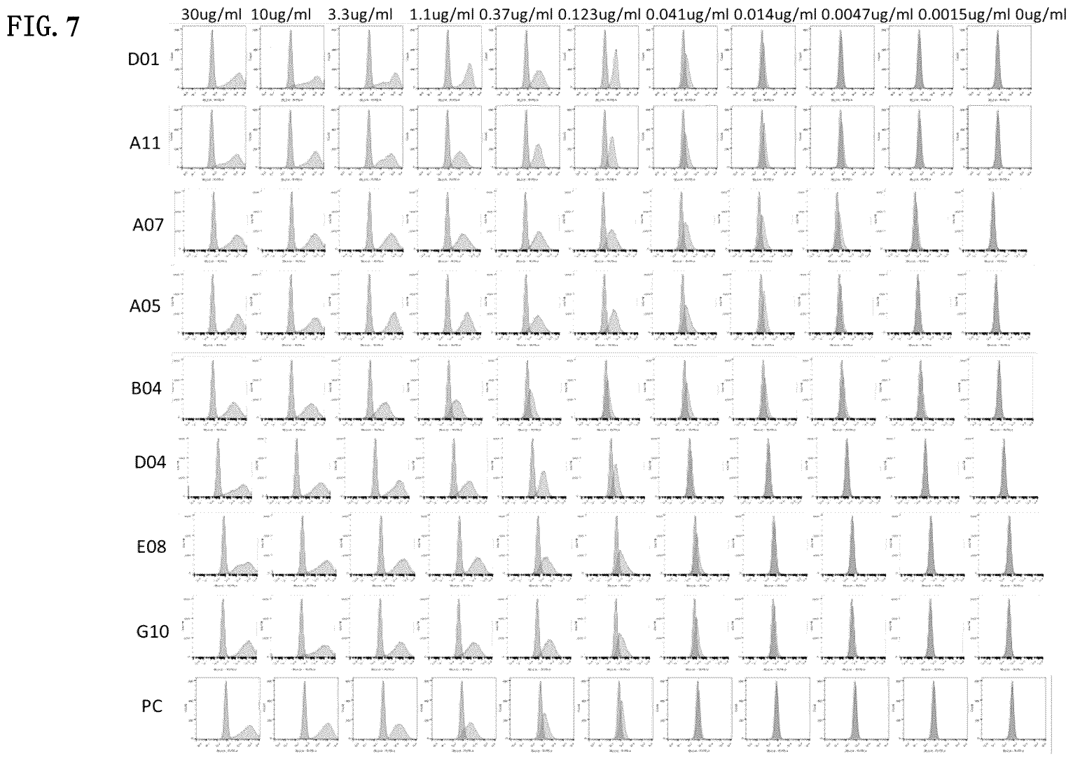

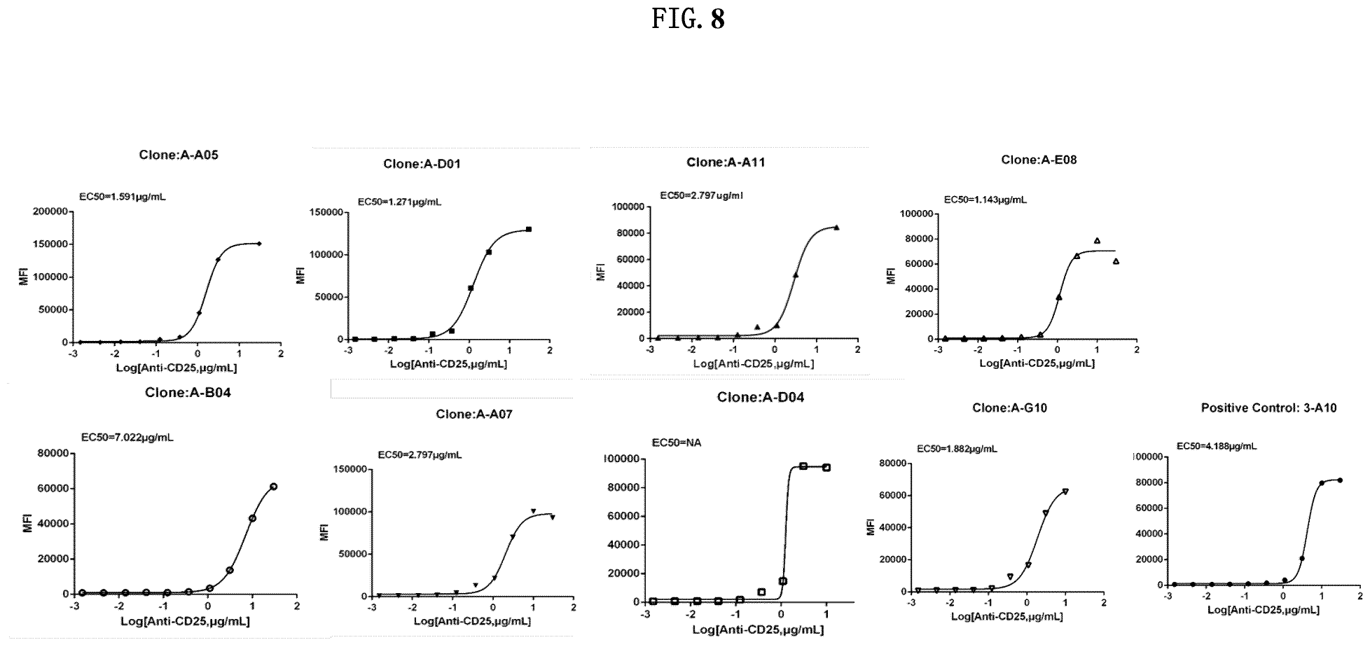

The results of the FACS binding EC₅₀s are as shown in FIG. 7 and FIG. 8.

The CHO-CD25-overexpressing cell line was resuscitated and adjusted to the logarithmic growth phase. After the candidate antibodies were transiently transfected into 293F cells for 48 hours, the supernatant was collected and FACS detection was performed. The results showed that the candidate antibodies could bind to the target membrane protein with high affinity, and the EC₅₀s were all better than that of the positive control antibody.

### 6. Functional Experiments on Candidate Antibodies

### ELISA Blocking Experiment

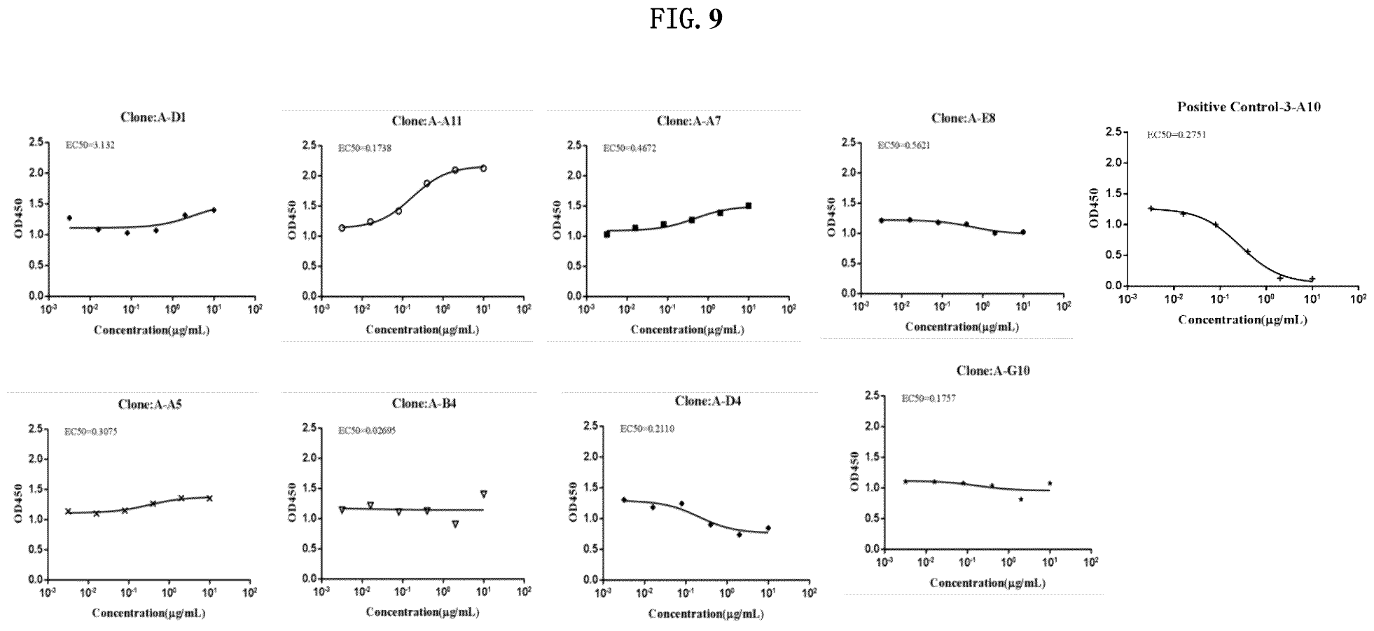

The CD25-His recombinant protein was coated on a microplate, and candidate antibodies at gradient concentrations and the Biotin-CD25 ligand protein were added. With the antibody concentration as the abscissa and the OD450 as the ordinate, ELISA was adopted to detect the blocking of the binding between the target protein and its ligand by the candidate antibodies, as shown in the following table. The results are as shown in FIG. 9. The results of the blocking experiment showed that the positive control antibody could significantly block the binding of the ligand to the CD25 protein, and there was a concentration gradient change; and none of the candidate antibodies significantly inhibited the binding of the CD25 protein to its ligand.

| Final | | | | | | | | |
|---|---|---|---|---|---|---|---|---|
| concentration | | | | | | | | |
| of positive | | | | | | | | |
| antibody | | | | | | | | |
| (μg/mL) | A-D1 | | A-A11 | | A-A7 | | A-A5 | |
| 10 | 1.437 | 1.37 | 2.114 | 2.144 | 1.466 | 1.547 | 1.306 | 1.403 |
| 2 | 1.311 | 1.334 | 2.117 | 2.081 | 1.422 | 1.355 | 1.359 | 1.361 |
| 0.4 | 1.127 | 1.081 | 1.91 | 1.845 | 1.335 | 1.205 | 1.254 | 1.282 |
| 0.08 | 1.033 | 1.031 | 1.466 | 1.374 | 1.159 | 1.233 | 1.166 | 1.137 |
| 0.016 | 1.091 | 1.08 | 1.265 | 1.217 | 1.151 | 1.126 | 1.08 | 1.128 |
| 0.0032 | 1.277 | 1.275 | 1.15 | 1.129 | 1.056 | 1.006 | 1.109 | 1.172 |
| 0 | 1.331 | 1.365 | 1.278 | 1.256 | 1.236 | 1.12 | 1.058 | 1.176 |
| PBS | 0.077 | 0.111 | 0.079 | 0.085 | 0.089 | 0.086 | 0.082 | 0.097 |

| Final | | | | | | | | | | |
|---|---|---|---|---|---|---|---|---|---|---|
| concentration | | | | | | | | | | |
| of antibody | | | | | | | | | Positive CD25 | |
| (μg/mL) | A-B4 | | A-D4 | | A-E8 | | A-G10 | | antibody | |
| 10 | 1.466 | 1.343 | 0.822 | 0.877 | 1.046 | 1 | 1.094 | 1.061 | 0.117 | 0.121 |
| 2 | 0.896 | 0.923 | 0.712 | 0.766 | 1.01 | 1.004 | 0.779 | 0.858 | 0.126 | 0.133 |
| 0.4 | 1.093 | 1.164 | 0.852 | 0.954 | 1.137 | 1.168 | 1.093 | 0.987 | 0.504 | 0.618 |
| 0.08 | 1.105 | 1.118 | 1.233 | 1.259 | 1.22 | 1.145 | 1.063 | 1.098 | 1.042 | 0.958 |
| 0.016 | 1.224 | 1.211 | 1.15 | 1.217 | 1.244 | 1.208 | 1.087 | 1.119 | 1.193 | 1.154 |
| 0.0032 | 1.074 | 1.215 | 1.27 | 1.344 | 1.249 | 1.183 | 1.15 | 1.065 | 1.308 | 1.22 |
| 0 | 1.197 | 1.228 | 1.219 | 1.513 | 1.347 | 1.252 | 1.155 | 1.142 | 1.309 | 1.307 |
| PBS | 0.076 | 0.077 | 0.075 | 0.079 | 0.078 | 0.078 | 0.079 | 0.079 | 0.081 | 0.077 |

The results of the ELISA blocking experiment showed that the positive control antibody could significantly block the binding of the ligand to the CD25 protein, and there was a concentration gradient change; and none of the candidate antibodies significantly inhibited the binding of the CD25 protein to its ligand.

### 7. Amino Acid Sequence Information of Candidate Antibody Clones

>A04
(SEQ ID NO: 2)
MAQVQLVESGGGLVQPGGSLRLSCAVSEVTSDSYAIGWFRQAPGKEREGVSCIST
TDGRTWYTDAVVGRFTISRDKAKNTVYLQMNSLKPDDTAVYYCGADGRDFCSAHVYS
RYPYDYSGQGTQVTVSS
>B10
(SEQ ID NO: 3)
MAEVQLVESGGGLVQPGGSLRLSCAVFGVTLDSYAIGWFRQAPGKEREGVSCIST
SDGRTWETDAVLGRFTISRDNAKNTVYLQMNDLKPEDTAVYYCGADGRDWCSAHVYS
RYEYDYSGQGTQVTVSS
>C10
(SEQ ID NO: 4)
MADVQLVESGGGLAQPGGSLTLSCATSGFTFSLYAMSWARLAPGEEIEWVSGIYS
DTNDTYYADSVKGRFAISRDNAKNTLYLQMNALKPEDTALYYCGAALERLCSSSMTCD
YWGQGTQVTVSS
>E07
(SEQ ID NO: 5)
MAQVQLVESGGGLVQPGGSLRLSCAASGFTFSSSAMSWARQAAPGKGLEWVSGI
YSDGSTYYADSMKGRFTISRDNAKNTVYLQMNSLKPEDTAVYYCAIGEWPPYDYWGQ
GTQVTVSS
>D07
(SEQ ID NO: 6)
MAQVQLVESGGGLVQPGGSLRLSCAASGFTFSSSAMSWARLAPGKEFEWVSGIY
SDGSTYYADSMEGRFTISRDNAKSTVYLQMNSLKPEDTAVYYCAIGPWPPYDYWGQGT
QVTVSS
>C05
(SEQ ID NO: 7)
MAAVQLVESGGGLAQPGGSLRLSCATSGFTFSSYAMSWARLAPGEELEWVSGVY
TDSNDTYYADSVKGRFAISRDNAKNTLYLQMNALKPEDTALYYCGAALGRLCSSSMTC
DYWGQGTQVTVSS
>D09
(SEQ ID NO: 8)
MAQVQLVESGGGSVQAGGSLKLSCVASGFTFSTYAMSWARLAPGEEIEWVSGIY
SDTNDTYYADSVKGRFAISRDNAKNTLYLQMNALKPEDTALYYCGAALGRLCSSSMTC
DYWGQGTQVTVSS
>D01
(SEQ ID NO: 9)
MAQVQLVESGGGLAQPGGSLRLSCAASGFTFSNFAVSWARQVAGEGFEWVSGV
YSDGDTYYADSVKDRFIISRDNAKNTLYLQMNSLKPEDTALYYCGAARGRLCSSIMTCD
YWGQGTQVTVSS
>A11
(SEQ ID NO: 10)
MADVQLVESGGGLVQPGGSLRLSCAASGFTFSSYAMSWYRQAPGKERELVAYIT
TSPGYTTYTDSVKGRFTISRDNAKNTVYLQMNSLKPDDTAVYYCNAWRGAPRGDYWG
QGTQVTVSS
>D04
(SEQ ID NO: 11)
MAQVQLVESGGGLVQPGGSLRLSCAASGFTFSSSAVSWARLTPGKRLEWVSAIAS
DGSTYYADSMQGRFTISRDNAKNTVYLQMNSLKPEDTAVYYCAIPRDGLVPVFGIDYW
GQGTQVTVSS
>E08
(SEQ ID NO: 12)
MAAVQLVESGGGLVQPGGSLRLSCAASGFTFSSYPMGWYRQAPGEECELVAGITS
AGGSTNYADSVKGRFTISRDNAKNTVYLQMNSLKPEDTAVYYCNTDPRYYTDSRGPCS
WGQGTQVTVSS
>G10
(SEQ ID NO: 13)
MADVQLVESGGGLAQPGGSLRLSCAASGFTFSNYAMNWLRQAPGKGLEWVSGI
YSDSSGTYYADSVKGRFTISRDNAKNTLYLQMNSLKPEDTALYYCAKASNSGSYYPWD
DDWGQGTQVTVSA
>A07
(SEQ ID NO: 1)
MAEVQLVESGGGLAQPGGSLRLSCAVSGFTLDYYAIGWFRQAPGKEREGVSCISG
SDGRTAYTDSVKGRFTISKDNAKNTVYLQMNSLKPEDTAVYYCAADPGYYCAGEVRYA
GSLYDYWGQGTQVTVSS
>A08
(SEQ ID NO: 14)
MAQVQLVESGGGLVQPGGSLRLSCAVSGFTLDYYAIGWFRQAPGKEREGVSCISG
SDGSTSYADSVKGRFTISRDNAKNTVYLQMNSLKPEDTAVYYCAADPGYYCSGYVRYA
GSLYDYWGQGTQVTVSS
>D08
(SEQ ID NO: 15)
MADVQLVESGGGLVQPGGSLRLSCAVSGFTLDYYAIGWFRQAPGKEREGVSCISG
SDGRTAYADSVKGRFTISKDNAKNTVYLQMNSLKPEDTAVYYCAADPGYYCAGEVRYA
GSLYDYWGQGTQVTVSS
>A05
(SEQ ID NO: 16)
MAQVQLVESGGGLAQPGGSLRLSCAASGFTFSIYAMSWARQAPGKGLEWVSGIY
SDSSYTYYADSVKGRFTISRDNAKNTLYLQMNSLKPEDTALYYCLKARGSGSYYPFDDY
RGQGTQVTVSS
>B04
(SEQ ID NO: 17)
MAQVQLVESGGGLVQPGGSLRLSCATSGFTLDYYAIGWFRQAPGKEREGVSCISS
SDGTTAYADSVKGRFTISTDNARNTVYLQMNSLKPEDTAVYYCAADLGYYCADNVRYA

### 8. Antibody Humanization

### 8.1 Humanization Design

According to the antibody screening results, clone A07 (SEQ ID NO:1) was selected for humanization design. The sequences of the designed humanized candidate antibodies are as follows:

>A07-HM1
(SEQ ID NO: 20)
EVQLVESGGGLVQPGGSLRLSCAVSGFTLDYYAIGWFRQAPGKGLEWVSCISGSD
GRTAYADSVKGRFTISKDNSKNTVYLQMNSLRAEDTAVYYCAADPGYYCAGEVRYAGS
LYDYWGQGTLVTVSS
>A07-HM2
(SEQ ID NO: 21)
EVQLLESGGGLVQPGGSLRLSCAVSGFTLDYYAIGWFRQAPGKGLEWVSCISGSD
GRTAYGDSVKGRFTISKDNSKNTVYLQMNSLRAEDTAVYYCAADPGYYCAGEVRYAGS
LYDYWGQGTLVTVSS
>A07-HM3
(SEQ ID NO: 22)
EVQLVESGGGVVQPGGSLRLSCAVSGFTLDYYAIGWFRQAPGKGLEWVSCISGS
DGRTAYADSVKGRFTISKDNSKNTVYLQMNSLRTEDTALYYCAADPGYYCAGEVRYAG
SLYDYWGQGTLVTVSS
>A07-HM4
(SEQ ID NO: 23)
EVQLVESGGGLVQPGGSLRLSCAVSGFTLDYYAIGWFRQAPGKGLEYVSCISGSD
GRTAYADSVKGRFTISKDNSKNTVYLQMGSLKPEDMAVYYCAADPGYYCAGEVRYAGS
LYDYWGQGTLVTVSS
>A07-HM5
(SEQ ID NO: 18)
EVQLVESGGGLVQPGGSLRLSCAVSGFTLDYYAIGWFRQAPGKGLEGVSCISGSD
GRTAYADSVKGRFTISKDNAKNTVYLQMNSLRAEDTAVYYCAADPGYYCAGEVRYAGS
LYDYWGQGTLVTVSS
>A07-HM6
(SEQ ID NO: 19)
EVQLLESGGGLVQPGGSLRLSCAVSGFTLDYYAIGWFRQAPGKGLEGVSCISGSD
GRTAYGDSVKGRFTISKDNAKNTVYLQMNSLRAEDTAVYYCAADPGYYCAGEVRYAGS
LYDYWGQGTLVTVSS

### 8.2 Detection of Humanized Antibodies

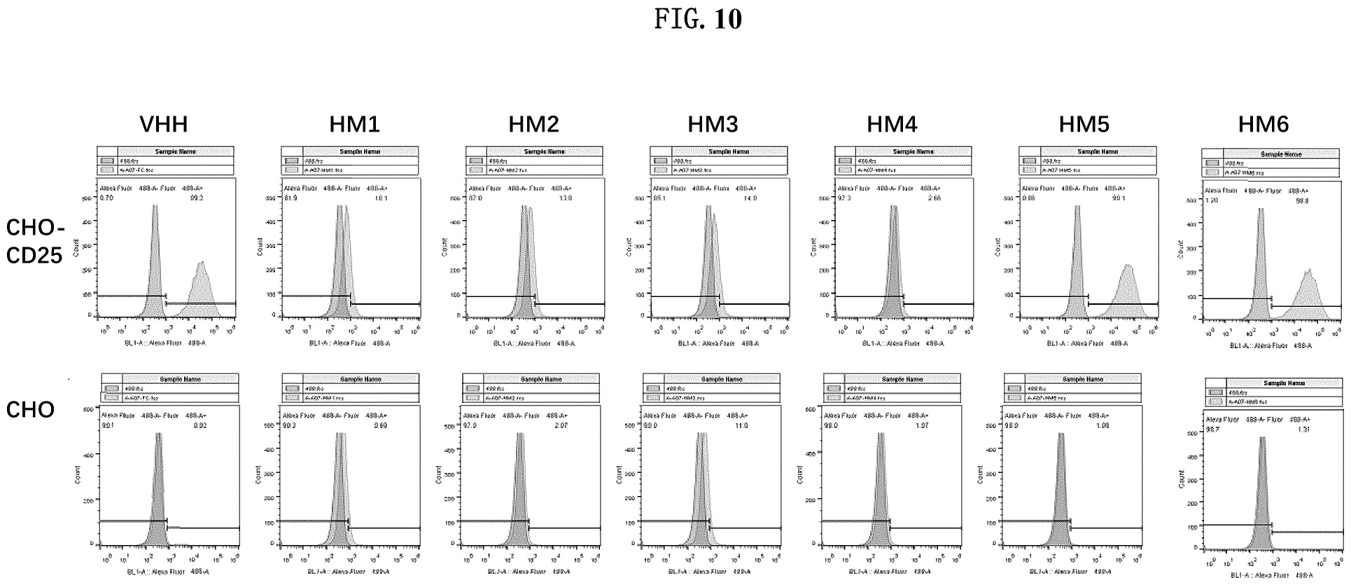

The preliminary FACS detection results are shown in FIG. 10.

According to the preliminary FACS detection results, the humanized clones HM5 and HM6 could bind to the target antigen. After further purifying the antibodies, the affinity detection was performed.

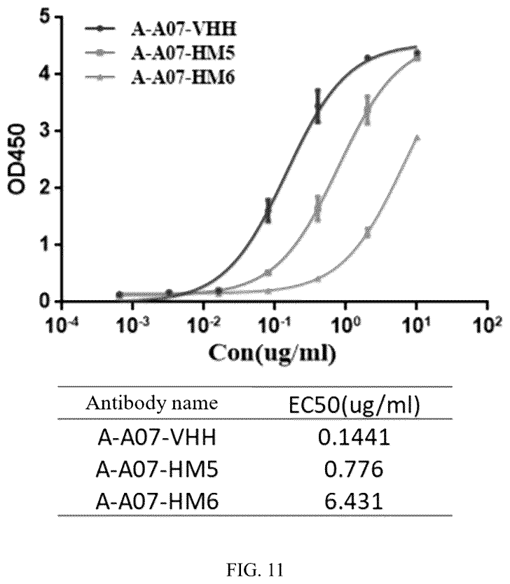

The graph illustrating the detection of the gradient dilution of humanized antibodies by ELISA is shown in FIG. 11.

| Antibody | | | | | | | |
|---|---|---|---|---|---|---|---|
| concentration | | A-A07-VHH | | A-A07-HM5 | | A-A07-HM6 | |
| 10 | μg/ml | 4.39 | 4.39 | 4.28 | 4.31 | 2.91 | 2.90 |
| 2 | μg/ml | 4.27 | 4.33 | 3.21 | 3.55 | 1.18 | 1.28 |
| 0.4 | μg/ml | 3.25 | 3.65 | 1.50 | 1.80 | 0.44 | 0.40 |
| 0.08 | μg/ml | 1.48 | 1.75 | 0.55 | 0.50 | 0.19 | 0.22 |
| 0.016 | μg/ml | 0.20 | 0.20 | 0.23 | 0.19 | 0.14 | 0.17 |
| 0.0032 | μg/ml | 0.18 | 0.16 | 0.16 | 0.17 | 0.18 | 0.20 |
| 0.00064 | μg/ml | 0.15 | 0.13 | 0.13 | 0.10 | 0.14 | 0.16 |

| | | | | | | |
|---|---|---|---|---|---|---|
| PBS | 0.14 | 0.14 | 0.14 | 0.14 | 0.14 | 0.16 |

After the gradient dilution of the candidate antibodies, the ELISA detection results showed that both of the two candidate humanized antibodies could efficiently bind to the target human CD25 antigen.

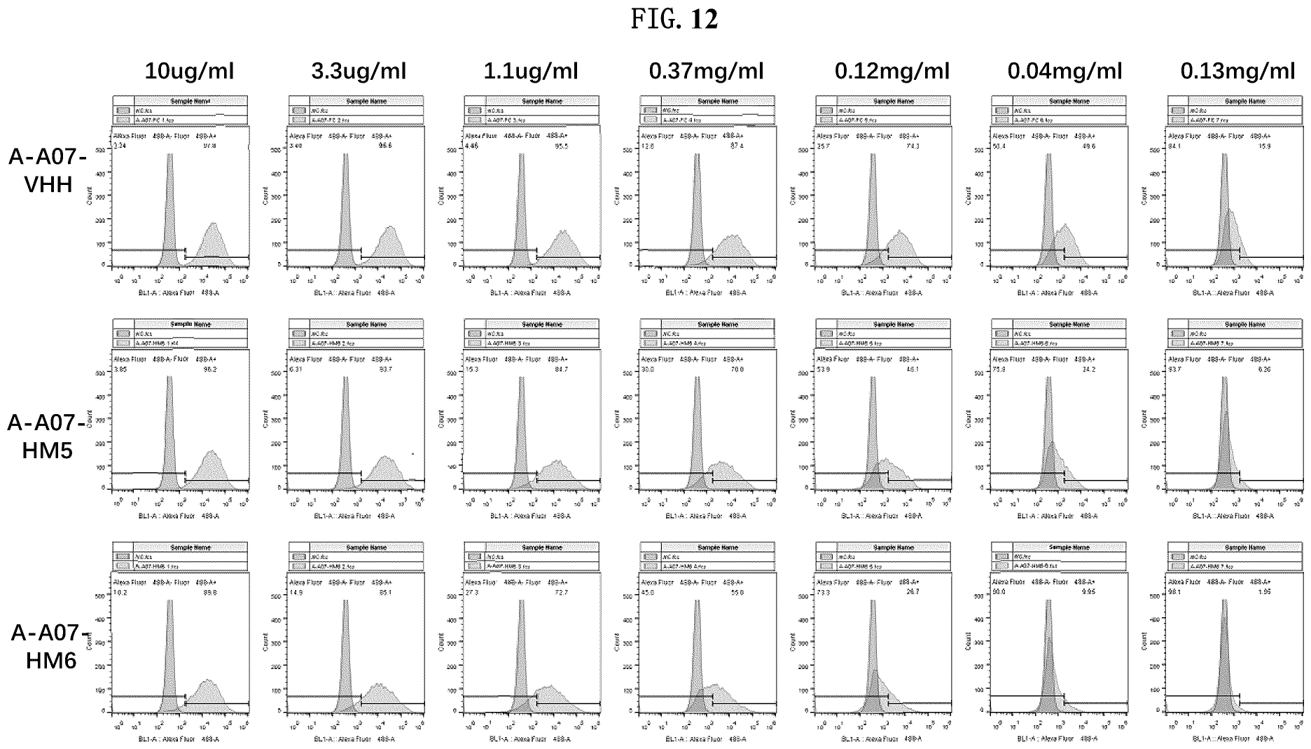

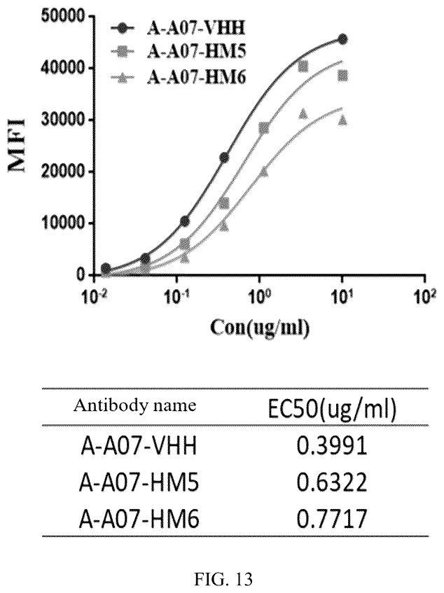

The detection results of the gradient dilution of humanized antibodies by FACS are shown in FIG. 12 and FIG. 13, which are consistent with the ELISA results. After the gradient dilution of the candidate antibodies, the FACS detection results showed that the candidate humanized antibodies could specifically bind to the human CD25 antigen expressed on the membrane.

### 9. ADCC Detection of Candidate Humanized Antibodies

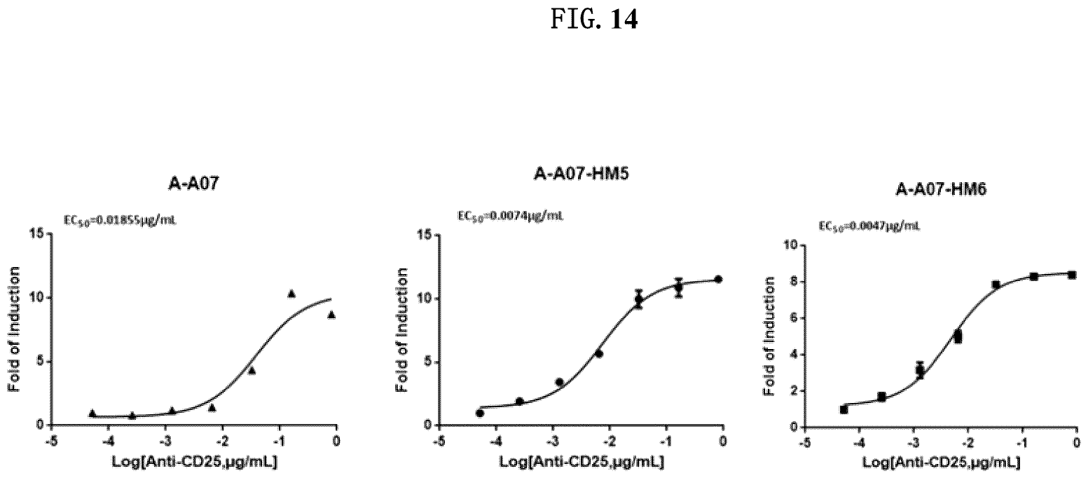

The detection results are shown in FIG. 14.

| Antibody | |
|---|---|
| name | EC₅₀ (μg/mL) |
| A-A07 | 0.01855 |
| A-A07-HM5 | 0.0074 |
| A-A07-HM6 | 0.0047 |

The ADCC experiments were performed on the original antibody before humanization and the two candidate humanized clones obtained after humanization. The results showed that the ADCC effect mediated by the candidate antibodies after humanization was improved.

### 10. Affinity Detection

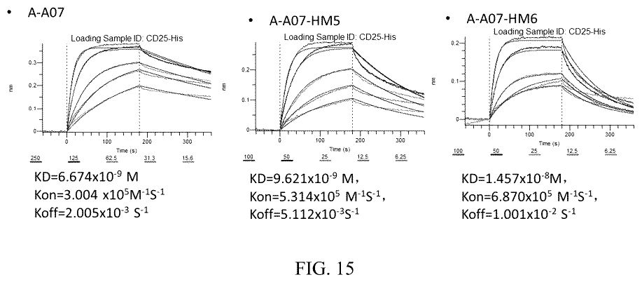

The detection results are shown in FIG. 15. The binding kinetic curve showed that the affinity of the candidate humanized antibody HM5 was equivalent to that of the original antibody.

### 11. ELISA IL-2 Blocking Experiment of Candidate Humanized Antibodies

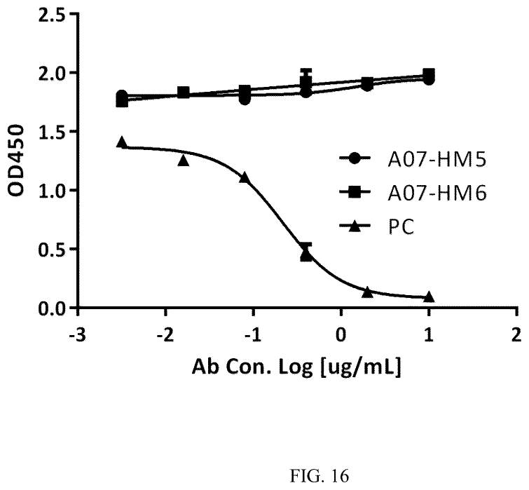

The CD25-His recombinant protein was coated on a microplate, and candidate antibodies at gradient concentrations and the Biotin-CD25 ligand protein IL-2 were added. With the antibody concentration as the abscissa and the OD450 as the ordinate, ELISA was adopted to detect the blocking of the binding between the target protein and its ligand by the candidate antibodies. The results are shown in FIG. 16.

| Antibody | | | | | | |
|---|---|---|---|---|---|---|
| concentration | | | | | Positive | |
| (μg/mL) | A07-HM5 | | A07-HM6 | | antibody | CD25 |
| 10 | 1.965 | 1.923 | 2.01 | 1.963 | 0.101 | 0.098 |
| 2 | 1.886 | 1.901 | 1.921 | 1.906 | 0.135 | 0.142 |
| 0.4 | 1.854 | 1.818 | 1.992 | 1.852 | 0.432 | 0.524 |
| 0.08 | 1.756 | 1.798 | 1.792 | 1.865 | 1.123 | 1.108 |
| 0.016 | 1.805 | 1.862 | 1.863 | 1.802 | 1.236 | 1.282 |
| 0.0032 | 1.799 | 1.815 | 1.775 | 1.738 | 1.436 | 1.399 |
| 0 | 1.901 | 1.823 | 1.906 | 1.912 | 1.862 | 1.9 |
| PBS control | 0.025 | 0.079 | 0.113 | 0.092 | 0.123 | 0.095 |

The results of the ELISA blocking experiment showed that the positive control antibody could significantly block the binding of the ligand to the CD25 protein, and there was a concentration gradient change; and none of the candidate humanized antibodies significantly inhibited the binding of the CD25 protein to its ligand.

### II. SUMMARY

In this project, multiple single-domain antibody sequences that specifically bind to the human CD25 antigen were developed, and clone A07 with the best binding effect was selected from these sequences for humanization modification. Finally, based on the affinity to the antigen and the ADCC effect, the candidate sequences of the humanized antibodies are selected as follows:

>A07-HM5
(SEQ ID NO: 18)
EVQLVESGGGLVQPGGSLRLSCAVSGFTLDYYAIGWFRQAPGKGLEGVSCISGSD
GRTAYADSVKGRFTISKDNAKNTVYLQMNSLRAEDTAVYYCAADPGYYCAGEVRYAG
SLYDYWGQGTLVTVSS
>A07-HM6
(SEQ ID NO: 19)
EVQLLESGGGLVQPGGSLRLSCAVSGFTLDYYAIGWFRQAPGKGLEGVSCISGSD
GRTAYGDSVKGRFTISKDNAKNTVYLQMNSLRAEDTAVYYCAADPGYYCAGEVRYA
GSLYDYWGQGTLVTVSS

In conclusion, the present invention provides a CD25 single-domain antibody, a humanized antibody and a preparation method thereof. The obtained CD25 single-domain antibody and humanized antibody have good binding performance, which can meet the needs of clinical applications and provides strong support for clinical applications.

The above are only the preferred examples of the present invention and are not intended to limit the present invention. Any modifications, equivalent substitutions, improvements, etc. made within the spirit and principles of the present invention shall be included in the scope of protection of the present invention.

What is claimed is:

1. A CD25 single-domain antibody, wherein a specific amino acid sequence is SEQ ID NO: 1.

2. A CD25 single-domain antibody, wherein a specific amino acid sequence is any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

3. A humanized antibody, wherein after humanization modification based on the CD25 single-domain antibody according to claim 1, a specific amino acid sequence is any one of SEQ ID NO: 18 and SEQ ID NO: 19.

4. A humanized antibody, wherein after humanization modification based on the CD25 single-domain antibody according to claim 1, a specific amino acid sequence is any one of SEQ ID NO: 20, SEQ ID NO: 21, SEQ ID NO: 22, and SEQ ID NO: 23.

5. A method for preparing a humanized CD25 single-domain antibody, comprising steps of:
Step 1: preparing a CD25 antigen;
Step 2: immunizing a Lama pacos with the CD25 antigen and detecting an immune titer;
Step 3: isolating a Lama pacos PBMC and obtaining a VHH antibody fragment;
Step 4: detecting affinity of a candidate antibody;
Step 5: performing a functional experiment on the candidate antibody;
Step 6: performing humanization modification of the antibody;
Step 7: detecting ADCC of a candidate humanized antibody;
Step 8: detecting binding affinity of the candidate humanized antibody to a target protein;
Step 9: performing an FACS experiment to detect binding of the candidate humanized antibody to an antigen-bearing cell; and
Step 10: performing an experiment of blocking binding of a ligand IL-2 by the candidate humanized antibody.

6. The method for preparing a CD25 single-domain antibody according to claim 5, further comprising: performing an FACS experiment for antigen-cell binding.

7. The method for preparing a CD25 single-domain antibody according to claim 6, further comprising: performing a functional experiment on ADCC of the candidate antibody.

8. The method for preparing a CD25 single-domain antibody according to claim 6, further comprising: performing an experiment of not interfering with the binding of the ligand IL-2.


SEQUENCE LISTING (23 sequences)

SEQ ID NO: 1 - protein, 130 aa, synthetic construct
MAEVQLVESGGGLAQPGGSLRLSCAVSGFTLDYYAIGWFRQAPGKEREGVSCISGSDGRTAYTDSVKGRFTISKDNAKNTVYLQMNSLKPEDTAVYYCAADPGYYCAGEVRYAGSLYDYWGQGTQVTVSS

SEQ ID NO: 2 - protein, 129 aa, synthetic construct
MAQVQLVESGGGLVQPGGSLRLSCAVSEVTSDSYAIGWFRQAPGKEREGVSCISTTDGRTWYTDAVVGRFTISRDKAKNTVYLQMNSLKPDDTAVYYCGADGRDFCSAHVYSRYPYDYSGQGTQVTVSS

SEQ ID NO: 3 - protein, 129 aa, synthetic construct
MAEVQLVESGGGLVQPGGSLRLSCAVFGVTLDSYAIGWFRQAPGKEREGVSCISTSDGRTWETDAVLGRFTISRDNAKNTVYLQMNDLKPEDTAVYYCGADGRDWCSAHVYSRYEYDYSGQGTQVTVSS

SEQ ID NO: 4 - protein, 125 aa, synthetic construct
MADVQLVESGGGLAQPGGSLTLSCATSGFTFSLYAMSWARLAPGEEIEWVSGIYSDTNDTYYADSVKGRFAISRDNAKNTLYLQMNALKPEDTALYYCGAALERLCSSSMTCDYWGQGTQVTVSS

SEQ ID NO: 5 - protein, 119 aa, synthetic construct
MAQVQLVESGGGLVQPGGSLRLSCAASGFTFSSSAMSWARQAAPGKGLEWVSGIYSDGSTYYADSMKGRFTISRDNAKNTVYLQMNSLKPEDTAVYYCAIGEWPPYDYWGQGTQVTVSS

SEQ ID NO: 6 - protein, 118 aa, synthetic construct
MAQVQLVESGGGLVQPGGSLRLSCAASGFTFSSSAMSWARLAPGKEFEWVSGIYSDGSTYYADSMEGRFTISRDNAKSTVYLQMNSLKPEDTAVYYCAIGPWPPYDYWGQGTQVTVSS

SEQ ID NO: 7 - protein, 125 aa, synthetic construct
MAAVQLVESGGGLAQPGGSLRLSCATSGFTFSSYAMSWARLAPGEELEWVSGVYTDSNDTYYADSVKGRFAISRDNAKNTLYLQMNALKPEDTALYYCGAALGRLCSSSMTCDYWGQGTQVTVSS

SEQ ID NO: 8 - protein, 125 aa, synthetic construct
MAQVQLVESGGGSVQAGGSLKLSCVASGFTFSTYAMSWARLAPGEEIEWVSGIYSDTNDTYYADSVKGRFAISRDNAKNTLYLQMNALKPEDTALYYCGAALGRLCSSSMTCDYWGQGTQVTVSS

SEQ ID NO: 9 - protein, 124 aa, synthetic construct
MAQVQLVESGGGLAQPGGSLRLSCAASGFTFSNFAVSWARQVAGEGFEWVSGVYSDGDTYYADSVKDRFIISRDNAKNTLYLQMNSLKPEDTALYYCGAARGRLCSSIMTCDYWGQGTQVTVSS

SEQ ID NO: 10 - protein, 120 aa, synthetic construct
MADVQLVESGGGLVQPGGSLRLSCAASGFTFSSYAMSWYRQAPGKERELVAYITTSPGYTTYTDSVKGRFTISRDNAKNTVYLQMNSLKPDDTAVYYCNAWRGAPRGDYWGQGTQVTVSS

SEQ ID NO: 11 - protein, 123 aa, synthetic construct
MAQVQLVESGGGLVQPGGSLRLSCAASGFTFSSSAVSWARLTPGKRLEWVSAIASDGSTYYADSMQGRFTISRDNAKNTVYLQMNSLKPEDTAVYYCAIPRDGLVPVFGIDYWGQGTQVTVSS

SEQ ID NO: 12 - protein, 124 aa, synthetic construct
MAAVQLVESGGGLVQPGGSLRLSCAASGFTFSSYPMGWYRQAPGEECELVAGITSAGGSTNYADSVKGRFTISRDNAKNTVYLQMNSLKPEDTAVYYCNTDPRYYTDSRGPCSWGQGTQVTVSS

SEQ ID NO: 13 - protein, 124 aa, synthetic construct
MADVQLVESGGGLAQPGGSLRLSCAASGFTFSNYAMNWLRQAPGKGLEWVSGIYSDSSGTYYADSVKGRFTISRDNAKNTLYLQMNSLKPEDTALYYCAKASNSGSYYPWDDDWGQGTQVTVSA

SEQ ID NO: 14 - protein, 130 aa, synthetic construct
MAQVQLVESGGGLVQPGGSLRLSCAVSGFTLDYYAIGWFRQAPGKEREGVSCISGSDGSTSYADSVKGRFTISRDNAKNTVYLQMNSLKPEDTAVYYCAADPGYYCSGYVRYAGSLYDYWGQGTQVTVSS

SEQ ID NO: 15 - protein, 130 aa, synthetic construct
MADVQLVESGGGLVQPGGSLRLSCAVSGFTLDYYAIGWFRQAPGKEREGVSCISGSDGRTAYADSVKGRFTISKDNAKNTVYLQMNSLKPEDTAVYYCAADPGYYCAGEVRYAGSLYDYWGQGTQVTVSS

SEQ ID NO: 16 - protein, 124 aa, synthetic construct
MAQVQLVESGGGLAQPGGSLRLSCAASGFTFSIYAMSWARQAPGKGLEWVSGIYSDSSYTYYADSVKGRFTISRDNAKNTLYLQMNSLKPEDTALYYCLKARGSGSYYPFDDYRGQGTQVTVSS

SEQ ID NO: 17 - protein, 130 aa, synthetic construct
MAQVQLVESGGGLVQPGGSLRLSCATSGFTLDYYAIGWFRQAPGKEREGVSCISSSDGTTAYADSVKGRFTISTDNARNTVYLQMNSLKPEDTAVYYCAADLGYYCADNVRYAGSLYDYWGQGTQVTVSS

SEQ ID NO: 18 - protein, 128 aa, synthetic construct
EVQLVESGGGLVQPGGSLRLSCAVSGFTLDYYAIGWFRQAPGKGLEGVSCISGSDGRTAYADSVKGRFTISKDNAKNTVYLQMNSLRAEDTAVYYCAADPGYYCAGEVRYAGSLYDYWGQGTLVTVSS

SEQ ID NO: 19 - protein, 128 aa, synthetic construct
EVQLLESGGGLVQPGGSLRLSCAVSGFTLDYYAIGWFRQAPGKGLEGVSCISGSDGRTAYGDSVKGRFTISKDNAKNTVYLQMNSLRAEDTAVYYCAADPGYYCAGEVRYAGSLYDYWGQGTLVTVSS

SEQ ID NO: 20 - protein, 128 aa, synthetic construct
EVQLVESGGGLVQPGGSLRLSCAVSGFTLDYYAIGWFRQAPGKGLEWVSCISGSDGRTAYADSVKGRFTISKDNSKNTVYLQMNSLRAEDTAVYYCAADPGYYCAGEVRYAGSLYDYWGQGTLVTVSS

SEQ ID NO: 21 - protein, 128 aa, synthetic construct
EVQLLESGGGLVQPGGSLRLSCAVSGFTLDYYAIGWFRQAPGKGLEWVSCISGSDGRTAYGDSVKGRFTISKDNSKNTVYLQMNSLRAEDTAVYYCAADPGYYCAGEVRYAGSLYDYWGQGTLVTVSS

SEQ ID NO: 22 - protein, 128 aa, synthetic construct
EVQLVESGGGVVQPGGSLRLSCAVSGFTLDYYAIGWFRQAPGKGLEWVSCISGSDGRTAYADSVKGRFTISKDNSKNTVYLQMNSLRTEDTALYYCAADPGYYCAGEVRYAGSLYDYWGQGTLVTVSS

SEQ ID NO: 23 - protein, 128 aa, synthetic construct
EVQLVESGGGLVQPGGSLRLSCAVSGFTLDYYAIGWFRQAPGKGLEYVSCISGSDGRTAYADSVKGRFTISKDNSKNTVYLQMGSLKPEDMAVYYCAADPGYYCAGEVRYAGSLYDYWGQGTLVTVSS